(19) United States
(12) Patent Application Publication
(10) Pub. No.: US 2006/0089414 A1
(43) Pub. Date: Apr. 27, 2006

(54) Generic probes for the detection of phosphorylated sequences

(76) Inventors: Kurt A. Morgenstern, Derry, NH (US); James P. Boyce, Kirkland, WA (US); Stewart D. Chipman, Bainbridge Island, WA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/186,852

(22) Filed: Jul. 22, 2005

Related U.S. Application Data
(60) Provisional application No. 60/590,705, filed on Jul. 23, 2004.

(51) Int. Cl. A61K 31/195; C07C 275/12

(52) U.S. Cl. 514/563; 562/560

(57) ABSTRACT

Generic probes that bind to phosphorylated amino acid residues are provided as well as methods employing the probes for screening for kinase inhibitory activity, kinase activity, and phosphatase activity. Methods for distinguishing serine/threonine kinase phosphorylation from tyrosine kinase phosphorylation are also provided.

This application claims the benefit of U.S. Provisional Application No. 60/590,705, filed Jul. 23, 2004, which is hereby incorporated by reference.

### DESCRIPTION OF THE INVENTION

Generic probes that bind to phosphorylated amino acid residues are provided as well as methods employing the probes for screening for kinase inhibitory activity, kinase activity, and phosphatase activity. Methods for distinguishing serine/threonine kinase substrate phosphorylation from tyrosine kinase substrate phosphorylation are also provided.

Screening for kinase inhibitors typically requires the detection of a phosphorylated substrate or substrates in a complex medium containing buffer components, salts, cofactors, proteins, peptide and small organic molecules. Radiometric assays are often used to directly screen for kinase activity in complex assay mediums. However, assay logistics, legal and safety issues make radiometric approaches less desirable than fluorescence-based assays for industrial-scale screening applications. Many fluorescence techniques, such as polarization, quenching, time correlation, and lifetime variation, that are based on intensity measurements, suffer from errors due to inner filter effects and the variability of the optical quality of the assay medium.

One fluorescence technique for high throughput kinase inhibitor screening is homogeneous time resolved fluorescence (HTRF) using fluorescence resonance energy transfer (FRET). This approach uses an energy donor-acceptor pair. Typically, europium crypate or europium chelate is the FRET donor and allophycocyanin (APC) is the FRET acceptor. The ratio of the FRET donor-acceptor signal is independent of the optical characteristics of the medium and depends predominantly on the specific biological interactions under study since the energy transfer efficiency depends on R₀, the inverse sixth power of the distance between the excited fluorescent donor and the acceptor molecule. The required distance R₀ between a FRET donor-acceptor pair for a 50% efficient energy transfer is generally 1-7 nm.

Currently, HTRF kinase inhibitor screening assays require a phosphoresidue- or phosphosubstrate-specific antibody to which a europium cryptate, europium chelate, or other lanthanide-based probe is covalently attached. The enzyme substrates are synthesized with biotin tags to enable a tight complex with allophytocyanin (APC)-strepavidin. Excitation of the europium-antibody bound to the phosphorylated substrate-APC complex results in FRET and the signal ratio of 665 nm:620 nm is determined to calculate the amount of substrate phosphorylation.

In addition to detecting substrate phosphorylation by protein kinases, substrate dephosphorylation by phosphatases can also be measured using FRET-based HTRF assays.

These current FRET-based assays were able to be developed based on the availability of high affinity and specific anti-phosphotyrosine antibodies, which are broadly applicable for screening the tyrosine family of kinases. However, the tyrosine family of kinase constitutes only approximately 25% of the entire superfamily of kinases. The serine/threonine kinase family represents a much larger percentage of the kinase superfamily, and accordingly serine/threonine kinase inhibitors are likely to afford a greater window of therapeutic opportunities. Accordingly, the ability to develop a generic assay to identify inhibitors of serine/threonine kinases is desirable.

However, antibodies with high affinity and specificity toward phosphoserine and phosphothreonine are difficult to generate. Most currently available anti-phosphoserine/phosphothreonine antibodies have suboptimal affinity and often cross-react with non-phosphorylated substrates. While a few antibodies have been successfully produced that bind to phosphoserine/phosphothreonine residues, they recognize phosphoserine/phosphothreonine only in the context of the residues flanking the phosphorylated residue. These reagents are not broadly applicable for screening the serine/threonine kinase family because substrate selectivity dictates the need for a unique antibody substrate pair for each kinase under study.

Accordingly, there is a need for new assay methods which are able to screen for kinase inhibitors of the entire kinase superfamily. Consequently, there is also a need for new generic probes that recognize phosphoserine and phosphothreonine residues as well as phosphotyrosine residues.

Generic probes that bind to phosphorylated amino acid residues are provided as well as methods employing the probes for screening for kinase inhibitory activity, kinase activity, and phosphatase activity. Methods for distinguishing serine/threonine kinase substrate phosphorylation from tyrosine kinase substrate phosphorylation are also provided.

One aspect of the present disclosure provides novel compounds having the formula:
C-D-E
wherein (C) is a coupling group, (D) is a linker group and (E) is a chelating group. These compounds may be coupled to fluorescence groups to form generic probes.

The coupling group (C) may be an electrophile, a nucleophile, or any radical that may be coupled to another molecule. For example, the coupling group (C) is chosen from an amino group, an aldehyde group, a C₁-C₆ alkyl halide group, a thiol group, and a hydroxy group. The amino group may be a primary amino group, i.e., —NH₂, or a secondary amino group, for example, having the structure —NHR′ wherein R′ is a C₁-C₆ alkyl group.

The linker group (D) is a bivalent radical. For example, the linker group (D) is chosen from:

—(CH₂)ₘ(OCH₂CH₂)ₙ—O—(CH₂)ₚ—(O)_{q}-Z-(CH₂)ᵣ—;

—(CR¹R²)ₘ—[(CR³R⁴)ₚ—(O)_{q}]ₙ-Z-(CR⁵R⁶)ᵣ—;

—(CH₂)ₘ—[(CR¹R²)ₚ—(O)_{q}]ₙ-Z-(CH₂)ᵣ—;

—(CH₂)ₘ—(C₆R¹R²R³R⁴)ₙ—(CH₂)ᵣ—; and

—(CH₂)ₘ—(CR¹, R²CR³R⁴NR⁵)ₙ—(CH₂)ₚ-Z-(CH₂)ᵣ—;

or (D) may be a linker group comprising at least one amino, aryl, or heteroaryl unit

wherein Z is a urea group or is absent;

m ranges from 0 to 3;

n ranges from 0 to 170;

p ranges from 0 to 3;

q is 0 or 1;

r ranges from 0 to 3; and

R¹, R², R³, R⁴, R⁵ and R⁶ are each independently chosen from hydrogen, fluorine, and C₁-C₆ alkyl; provided that

when Z is absent, n is 0, and the chelating group (E) is of the formula: 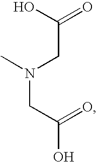
then m, p, and q are each not 2.

The chelating group (E) is a phosphate modifying group, such as a radical that is capable of binding to a modified or unmodified phosphate group, for example, a radical that binds to a metal atom and forms a complex with the phosphate group. For example, the chelating group (E) may be chosen from a thiol, an imidazo group, a hydroxamic acid group, a hydroxylamine group, and a sulfonic acid group.

In some embodiments, R¹ and R² of the linker group (D) are each hydrogen. In other embodiments, R¹, R², R³, and R⁴ are each hydrogen. In yet other embodiments, R¹, R², R³, R⁴, R⁵ and R⁶ are each hydrogen.

In some embodiments, at least one of m, p, and q of the linker group (D) ranges from 1 to 3. In other embodiments the sum of m, n, p, and r ranges from 0 to 170 if Z is present or from 1 to 170 if Z is not present. In yet other embodiments, n ranges from 1 to 125, 1 to 100, 1 to 75, 1 to 50, 1 to 20, or even 1 to 5, such as 2.

In some embodiments, Z of the linker group (D) is a urea group, for example, having the formula —NHC(O)NH— or —CH₂CH₂NHC(O)NH—. In other embodiments, Z is absent.

In some embodiments, the compounds C-D-E have the following formula: 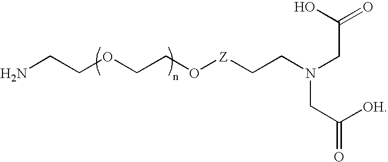
In some of these embodiments, Z is a urea group, for example, —CH₂CH₂NHC(O)NH—, or may be absent.

In some embodiments, the chelating group (E) is of the formula:

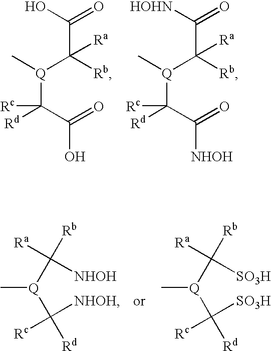

wherein Q is chosen from N, P, and CH, and

R^{a}, R^{b}, R^{c}, and R^{d} are each independently chosen from hydrogen, fluorine, and C₁-C₆ alkyl. Alternatively, one or both of (R^{a} and R^{b}) or (R^{c} and R^{d}) may together form a carbonyl group. In some embodiments, R^{a}, R^{b}, R^{c}, and R^{d} are each hydrogen. In some embodiments, Q is N. In certain of these embodiments, Q is N and R^{a}, R^{b}, R^{c}, and R^{d} are each hydrogen.

In other embodiments, the chelating group (E) is of the formula:

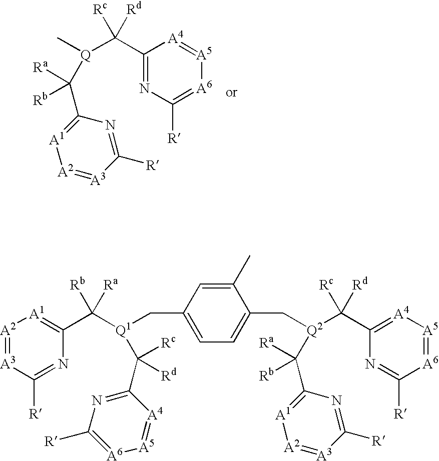

wherein each Q (including Q¹ and Q²) is chosen from N, P, and CH;

R^{a}, R^{b}, R^{c}, and R^{d} are each independently chosen from hydrogen, fluorine, and C₁-C₆alkyl; and

A¹, A², A³, A⁴, A⁵, and A⁶ are each independently chosen from N and C—R′, wherein each R′ is chosen from hydrogen, fluorine and C₁-C₆ alkyl. These chelating groups may bind to phosphate groups at pHs ranging from 6 to 8, such as neutral pH (7). In some embodiments, Q (or one or both of Q¹ and Q²) is N; R^{a}, R^{b}, R^{c}, and R^{d} are each hydrogen; and A¹, A², A³, A⁴, A⁵, and A⁶ are each CH.

In one embodiment, the compound: 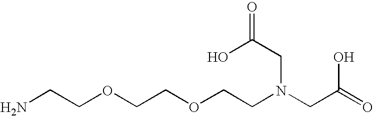
is provided. In another embodiment, the compound: 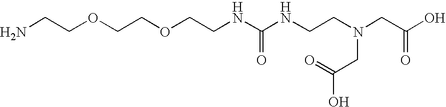
is provided. Yet in other embodiments, the following compounds are provided:

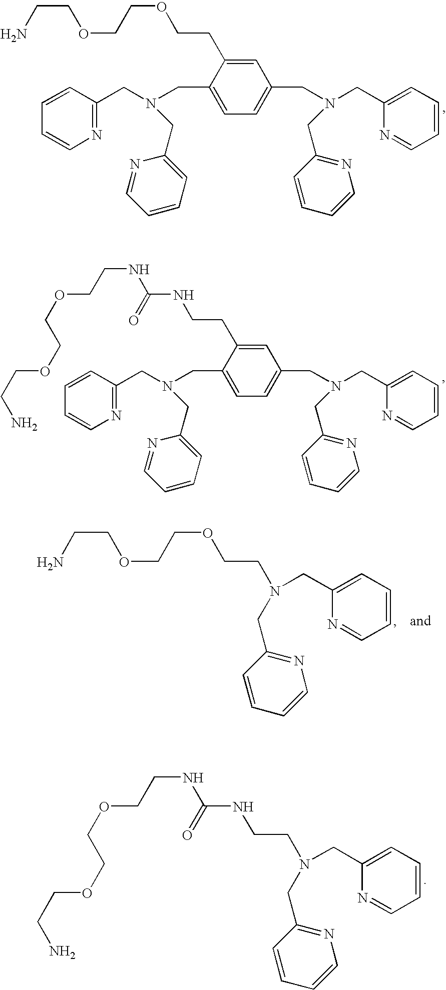

Another aspect of the present disclosure provides novel compounds having the formula:
A-B′—C′-D-E
wherein (A) is a fluorescence group, (B′) is a residue of a first coupling group, (C′) is a residue of a second coupling group, (D) is a linker group, and (E) is a chelating group. These compounds are useful as generic probes.

The fluorescence group (A) is any radical capable of emitting fluorescent energy. The fluorescence group (A) may be chosen from metal chelates, metal cryptates, and fluorescence groups, including fluorescence donor groups. In certain embodiments, the fluorescence group (A) may be any haptan (e.g., phosphotyrosine, dinitrophenol, and fluorescein) that is capable of being bound by a second probe to form the fluorescence group.

The residue of a first coupling group (B′) and the residue of a second coupling group (C′) are each independently chosen from an amino group, a carbonyl group, a C₁-C₆ alkyl group, a sulfur atom, and an oxygen atom. These groups are, respectively, the residues of an amino group, an aldehyde group, a C₁-C₆ alkyl halide group, a thiol group, and a hydroxy group. One of skill in the art will recognize that the residues of the first and second coupling groups (B′) and (C′) are chosen such that a compatible coupling reaction can occur. For example, when the first coupling group (B) is an amino group —NH₂, and the second coupling group (C) is an aldehyde group, the residue of the first coupling group (B′) is —NH— and the residue of the second coupling group (C′) is carbonyl such that (B′) and (C′) together form an amide group. Similarly, (B′) and (C′) together form an amide group also when (B′) is a carbonyl and (C′) is an amide.

The linker group (D) and chelating group (E) are as described above.

In some embodiments, the fluorescence group (A) is a metal chelate or metal cryptate. The metal may be chosen from transition metals, lanthanide elements, and actinide elements such as europium, gadolinium, terbium, zinc, ruthenium and thorium. In some embodiments, the fluorescence group (A) is a fluorescence group. In other embodiments, the fluorescence group (A) is a metal chelate or a metal cryptate, for example, a rare earth metal cryptate.

In other embodiments, the fluorescence group (A) is a macrocyclic rare earth metal complex. Such macrocyclic rare earth metal complexes are described in U.S. Pat. No. 5,457,184. One group of macrocyclic rare earth metal complexes have the following formula: 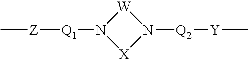
in which the bivalent radicals W, X, Y, and Z, which are identical or different, are hydrocarbon chains optionally containing one or more heteroatoms, at least one of the radicals containing at least one molecular unit or essentially consisting of a molecular unit possessing a triplet energy greater than the energy of the emission level of the complexed rare earth ion, at least one of said radicals consisting of a substituted or unsubstituted nitrogen-containing heterocyclic system in which at least one of the nitrogen atoms carries an oxy group, and wherein one or both of the radicals Y and Z optionally is not present; and
Q₁ and Q₂, which are identical or different, are either hydrogen (in which case one or both radicals Y and Z do not exist), or a hydrocarbon chain, e.g., (CH₂)₂, optionally interrupted by one or more heteroatoms, n being an integer from 1 to 10.

One embodiment includes the proviso that if the radicals W and/or X are a nitrogen-containing heterocyclic system in which at least one of the nitrogen atoms carries an oxy group, the radicals Y and/or Z are selected from biquinolines, biisoquinolines, bipyridines, terpyridines, coumarins, bipyrazines, bipyrimidines and pyridines.

In some embodiments, the macrocyclic rare earth complexes comprise at least one rare earth salt complexed by a macrocyclic compound of the formula above in which at least one of the bivalent radicals W and X contains at least one molecular unit or essentially consists of a molecular unit possessing a triplet energy greater than the energy of the emission level of the complexed rare earth ion, and at least one of the radicals Y and Z consists of a nitrogen-containing heterocyclic system in which at least one of the nitrogen atoms carries an oxy group.

In certain embodiments, the macrocyclic rare earth metal complexes described above, W and X are identical, Y and Z are identical, and/or Q₁ and Q₂ are identical. Some of these embodiments include the proviso if the radicals W and/or X are a nitrogen heterocyclic system in which at least one of the nitrogen atoms carries an oxy group, the radicals Y and/or Z are selected from biquinolines, biisoquinolines, bipyridines, terpyridines, coumarins, bipyrazines, bipyrimidines and pyridines.

In certain embodiments, Q₁, Q₂, W, X, Y, and Z are each independently chosen from phenanthroline; anthracene; bipyridines; biquinolines, such as bisisoquinolines, for example 2,2′-bipyridine; terpyridines; coumarins; bipyrazines; bipyrimidines; azobenzene; azopyridine; pyridines; 2,2′-bisisoquinoline, as well as the units:

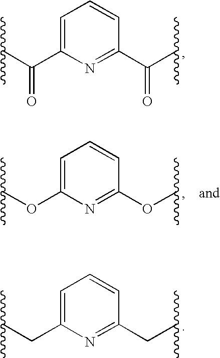

In some embodiments, the nitrogen-containing heterocyclic system in which at least one of the nitrogen atoms carries an oxy group is chosen from pyridine N-oxide, bipyridine N-oxide, bipyridine di-N-oxide, bisisoquinoline-N-oxide, bisisoquinoline di-N-oxide, bipyrazine N-oxide, bipyrazine di-N-oxide, bipyrimidine N-oxide, and bipyrimidine di-N-oxide.

These macrocyclic rare earth metal complexes may be complexed with rare earth ions such as terbium, europium, samarium and dysprosium ions.

The triplet energy-donating molecular units possess a triplet energy greater than or equal to the energy of the emission levels of the rare earth ion, for example, greater than 17,300 cm⁻¹.

The macrocyclic rare earth metal complexes may be substituted at least one of groups W, X, Y, and Z by a group —CO—NH—R″—R′″ in which R″ is a spacer arm or group which comprises or consists of a bivalent organic radical selected from linear or branched C₁ to C₂₀ alkylene groups optionally containing one or more double bonds and/or optionally interrupted by one or more heteroatoms such as oxygen, nitrogen, sulfur or phosphorus, from C₅ to C₈ cycloalkylene groups or from C₆ to C₁₄ arylene groups, the alkylene, cycloalkylene or arylene groups optionally being substituted by alkyl, aryl or sulfonate groups; and R′″ is a functional group capable of bonding covalently with a biological substance such as NH₂, COOH, SH, and OH.

In certain embodiments, the cryptate is a trisbipyridine cryptate. In some of these embodiments, the fluorescence group (A) and the first coupling group (B) together have a formula chosen from: 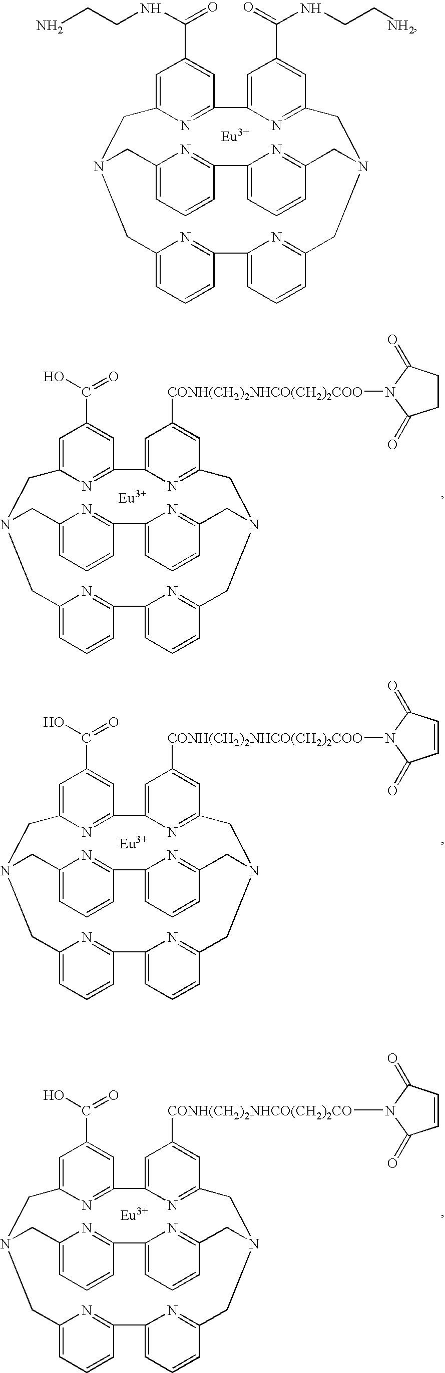 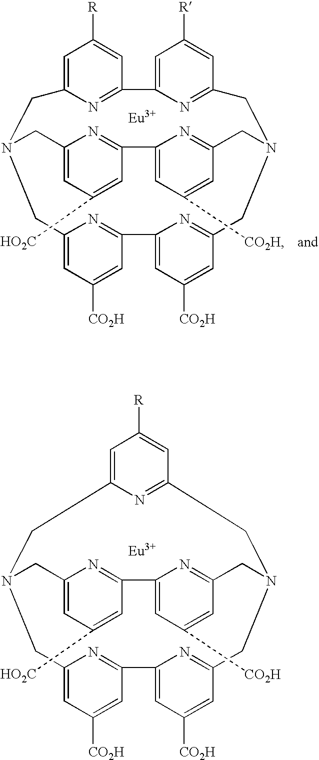
wherein each R is —C(O)NH(CH₂)₂NH,

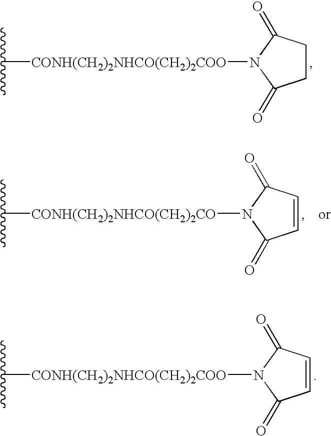

Accordingly, resulting the fluorescence group (A) and the residue of the first coupling group (B′) are together have a formula chosen from: 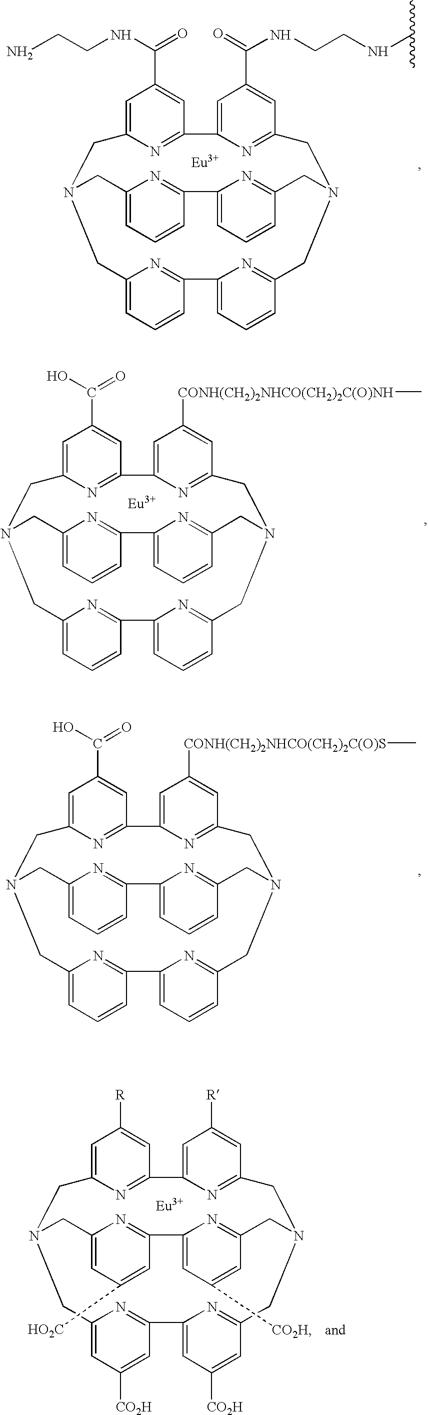 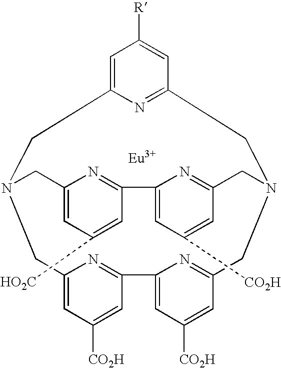
wherein each R is —C(O)NH(CH₂)₂NH, , 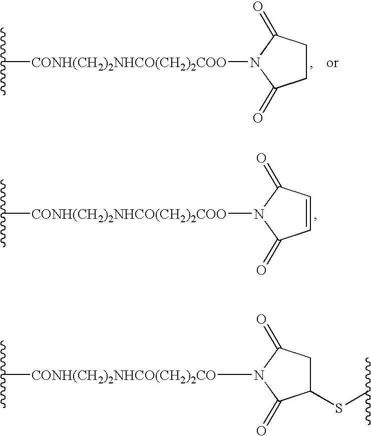
and R′ is —C(O)NH(CH₂)₂NH— or —C(O)NH(CH₂)₂S—.

In certain embodiments, the cryptate is a pyridine bipyridine cryptate. In some of these embodiments, the fluorescence group (A) and the residue of a first coupling group (B) together have a formula chosen from: 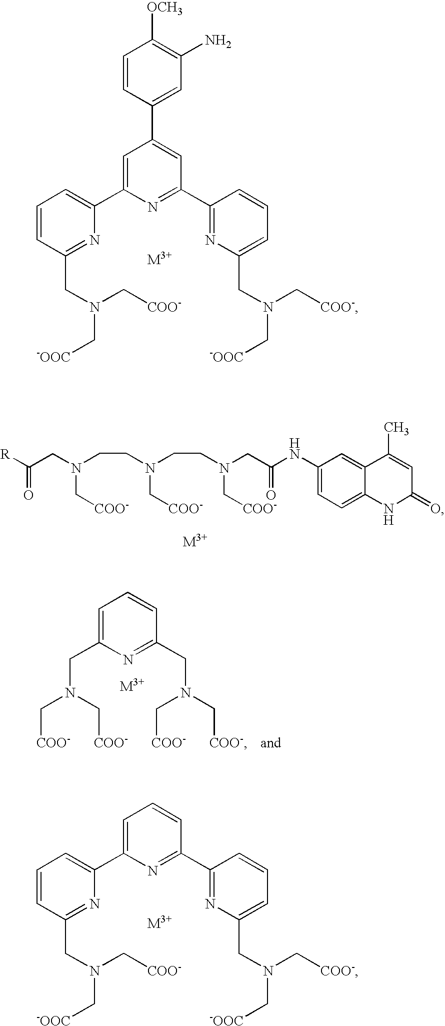
wherein M³⁺ is chosen from Eu³⁺ and Tb³⁺. U.S. Pat. Nos. 4,925,804; 5,637,509; 4,761,481; 4,920,195; 5,032,677; 5,202,423; 5,324,825; 5,457,186; and 5,571,897 as well as PCT Publication No. WO 87/07955, also disclose examples of molecules that may be used to form the fluorescence group (A) and the residue of a first coupling group (B′).

Another aspect of the present disclosure provides novel compounds having the formula:
A-B′—C′-D-E-F-G
wherein (A) is a fluorescence group, (B′) is a residue of a first coupling group, (C′) is a residue of a second coupling group, (D) is a linker group, (E) is a chelating group, (F) is a metal, and (G) is a phosphopeptide or phosphoprotein. The fluorescence group (A), residue of a first coupling group (B′), residue of a second coupling group (C′), linker group (D), and chelating group (E) are as described above. These compounds are formed when generic probes bind to a phosphate residue of a phosphopeptide or phosphoprotein.

The metal (F) may be chosen from is metal and may be a cation. These cations include, but are not limited to, Fe³⁺, Ga³⁺, Ru²⁺, Th³⁺, Zn²⁺, Zr²⁺, Zr³⁺, and Ni⁺.

The phosphopeptide or phosphoprotein (G) may comprise one or more of a phosphothreonine residue, a phosphoserine residue, or a phosphotyrosine residue. The phosphopeptide or phosphoprotein (G) may be mono- or polyphosphorylated. In certain embodiments, the phosphopeptide or phosphoprotein (G) has just one phosphorylated residue. The phosphopeptide or phosphoprotein (G) may be biotinylated.

In another aspect, the disclosure provides compounds of the formula:
A-B—C′-D-E-F-G′
wherein (A) is a fluorescence group, (B′) is a residue of a first coupling group, (C′) is a residue of a second coupling group, (D) is a linker group, (E) is a chelating group, (F) is a metal, and (G′) is a peptide or protein comprising at least four histidine residues. The fluorescence group (A), residue of a first coupling group (B′), residue of a second coupling group (C′), linker group (D), and chelating group (E) are as described above. These compounds are formed when generic probes bind to proteins or peptides comprising at least four histidine residues, e.g., His-tagged proteins or peptides.

The metal (F) may be a cation. One such cation is nickel, e.g., Ni²⁺.

The peptide or protein (G′) comprises at least four histidine residues and may be phosphorylated or not phosphorylated. In some embodiments, the peptide or protein (G′) comprises six or more histidine residues. The histidine residues may be contiguous or close to each other in space in the case of a folded protein.

In another aspect of the present disclosure, bivalent compounds of the formula: 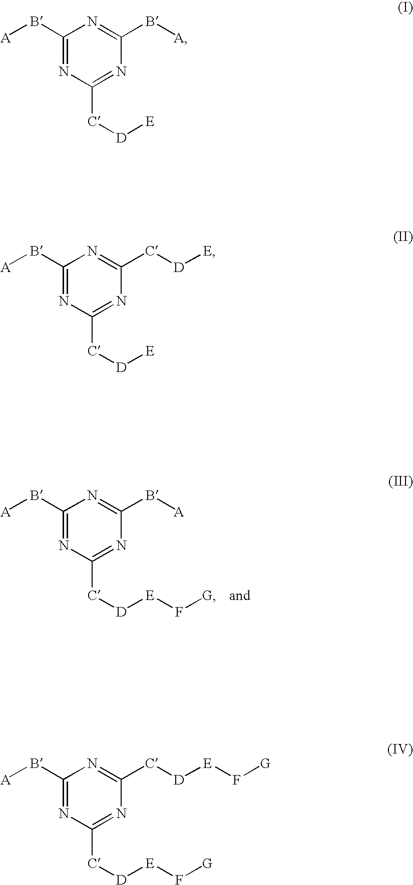
are provided wherein the groups (A), (B′), (C′), (D), (E), (F), and (G) are as described above. One of skill in the art will recognize that compound (I) is a probe with two fluorescent groups, and forms compound (III) when bound to a phosphopeptide or phosphoprotein ligand. Compound (I) emits more fluorescence per ligand than the A-B′—C′-D-E probes described above because there are two fluorescence groups (A). Compound (II) is a probe with two ligand binding sites and forms compound (IV) when bound to two ligands. Accordingly, compound (II) emits less fluorescence per ligand as the A-B′—C′-D-E probes described above. Although compounds (III) and (IV) are illustrated with peptides or proteins (G), one of skill in the art will recognize that probes (I) and (II) may also bind peptides or proteins comprising at least four histidine residue (G′).

Any of the probes described above may be coupled to a solid support to allow for easy separation, for example, via a linker.

In another aspect, kinase activity assays are provided. In one embodiment, methods for identifying kinase activity of a test protein are provided which comprise preparing an assay medium comprising a test protein, optionally a second protein or peptide, a metal ion, and a compound of the formula A-B′—C′-D-E as described above, exciting the assay medium at a first wavelength; measuring a fluorescence intensity of the assay medium at a second wavelength; and determining the kinase activity of the test protein using the fluorescence intensity of the assay medium.

The first wavelength may be an excitation wavelength of the fluorescence group (A), for example, ranging from 300 to 330 nm. The second wavelength may range from 580 to 720 nm, for example, 665 nm. One of skill in the art can readily determine the optimal excitation and emission wavelengths for the fluorescence group (A) employed in the assay.

The assay medium may be a solution and may optionally comprise at least one of ATP, a buffer (such as HEPES), dithiothreitol (DTT), bovine serum albumin (BSA), and salts (e.g., NaCl, MgCl₂ and MnCl₂), and cofactors. Alternatively, the assay medium may be on plates, wells, membranes, filters, beads, gels, and the like.

While not wishing to be bound by theory, it is believed that the probes form metal coordination complexes with the phosphate groups of the phosphopeptides and phosphoproteins. For example, the scheme below shows a probe coupled to a solid support bind to a metal atom, Fe³⁺, and then bind to a phosphopeptide.

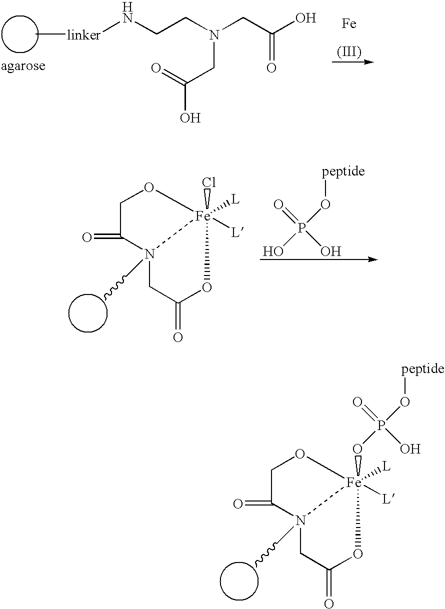

The second protein or peptide may comprise at least one phosphothreonine residue, allowing for identification of the test protein as a serine/threonine kinase. Similarly, the second protein or peptide may comprise at least one phosphoserine residue, allowing for identification of the test protein as a serine/threonine kinase. Alternatively, the second protein or peptide may comprise at least one phosphotyrosine residue, allowing for identification of the test protein as a tyrosine kinase.

In one embodiment, the test protein is a kinase that is capable of autophosphorylation.

In another embodiment, methods for identifying serine/threonine kinase phosphorylation are provided. Generally, the methods comprise performing the assay as described above to determine the total phosphorylation, performing an art-known assay to determine the tyrosine phosphorylation, for example, using a technique with an anti-phosphotyrosine antibody, and subtracting the tyrosine phosphorylation from the total phosphorylation to calculate the serine/threonine phosphorylation of the kinase. This analysis may also be used to distinguish serine/threonine phosphorylation and tyrosine phosphorylation.

Generally, methods for identifying kinase inhibitory activity of a test molecule are provided comprising preparing an assay medium comprising the test molecule, a kinase, a peptide, a metal ion, and a compound of the formula A-B′—C′-D-E as described above; exciting the assay medium at a first wavelength; measuring the fluorescence intensity of the assay medium at a second wavelength; calculating the kinase activity of the kinase using the fluorescence intensity of the assay medium; and determining the kinase inhibitory activity of the test molecule using the calculated kinase activity. Those of skill in the art will appreciate that this method may be adapted to identify kinase activity of more than one test molecule, for example, as a high-throughput assay.

The peptide is a phosphopeptide comprising at least one of a phosphothreonine residue, a phosphoserine residue, and a phosphotyrosine residue, allowing for identification of serine/threonine kinase inhibitors and/or tyrosine kinase inhibitors.

One example of a method for identifying kinase inhibitory activity of a test molecule (or test inhibitor) may be performed is as follows: In a 96-well plate, 50-200 μl of the following assay medium is added: 50 mM Hepes (pH 7.5), 0-250 mM NaCl, 0-5 mM DTT, 0-1% BSA, 0-200 mM MgCl₂, 0-200 mM MnCl₂, kinase substrate, cofactors (if required), ATP, test inhibitor(s), and enzyme. A control assay medium is set up in the same way but omitting the test inhibitor(s) and a blank assay medium is set up as described above, but with the addition of 0.1 to 0.5 M EDTA to inhibit the enzyme. One of skill in the art can readily determine concentrations of each reaction component for each kinase to achieve the desired activity. The kinase substrate and ATP are then added at concentrations incremental to the Kₘ values, which are previously determined by varying the concentration of each separately until saturation is achieved. The kinase substrate may be any molecule to which an affinity tag, such as biotin, is attached such as includes proteins, lipids, and peptide sequences. For peptide substrates, the biotin is typically attached to the N-terminal residue and the total length of the peptide ranges from 6 to 20 amino acids. The distance between the biotin affinity tag and the phosphorylation site typically ranges from 1 to 15 residues, for example, from 1 to 8. The assay reaction contains molecules to be tested for kinase inhibitor properties which are titrated from a stock solution of DMSO such that the final DMSO concentration is below a level that does not dramatically alter enzyme activity relative to the control assay in the absence of DMSO. Inhibitor concentrations typically range from 0 to 20 μM. The reactions with and without inhibitors are incubated for an amount of time that is linearly related to the catalytic turnover of substrate in the absence of inhibitor. The assay may also be performed on microchips, or other well plates, for example, 384 or 1536 well plates.

The probe may be coupled to a solid support, for example, via a linker, to facilitate separation of phosphoproteins and phosphopeptides from the assay medium.

The kinase products may be detected as follows: The enzyme reactions are quenched by addition of quench buffer containing from 0.1 to 0.5 M EDTA and from 0.1 to 0.5 M KF. This is followed by the addition of APC (allophycocyanin)-streptavidin for a predetermined incubation time (˜1-2 hours) to assure saturation of the biotin tagged substrates. The APC-streptavidin:biotin ratio is empirically determined at predefined enzymatic conditions to yield an optimal signal. Acid is added to reduce the pH to between 2 and 5 followed by the addition of a predetermined concentration of the europium cryptate conjugated probe (A-B′—C′-D-E). The probe:ATP ratio is predetermined since some nonspecific binding to ATP may occur. The detection reagents are incubated for 4 to 6 hours. Specific FRET may be read at both 665 nm and 620 nm using a RubyStar reader. To minimize medium interference, the ratio of fluorescence at 665 and 620 is calculated. Specific FRET is expressed as % AF as follows:

$$\frac{665 nm \text{/} 620 nm ( Sample ) - 665 nm \text{/} 620 nm ( Blank )}{665 nm \text{/} 620 nm ( Control ) - 665 nm \text{/} 620 nm ( Blank )} \times 100 = \% Δ F = \% Inhibition$$

wherein the sample has enzyme and inhibitor (DMSO) and the reaction is quenched at 90 min, the control has the enzyme without inhibitor (DMSO) and the reaction is quenched at 90 min. and the blank has the enzyme without inhibitor (DMSO) and the reaction is quenched at 0 min.

The probes described above may also be employed in methods to identify phosphatase activity and inhibition of phosphatase activity, including phosphoserine/phosphothreonine phosphatases, phosphotyrosine phosphatases, and mixed phosphatases. Those of skill in the art can readily adopt the methods described above for this purpose, which generally involves substituting a phosphatase for the kinase enzyme and providing a phosphorylated substrate. The buffer conditions may be varied with no more than routine skill, for example, by not including ATP.

It is to be understood that both the foregoing general description and the following detailed description are exemplary and explanatory only and are not restrictive of the invention, as claimed.

Other embodiments of the invention will be apparent to those skilled in the art from consideration of the specification and practice of the invention disclosed herein. It is intended that the specification and examples be considered as exemplary only.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in its respective testing measurements.

### EXAMPLES

### Example 1

### Synthesis of a C-D-E Compound

Compound 5 (Senn Chem, Inc.) was alkylated with benzyl-2-bromacetate (DIEA/THF/H₂O) at room temperature (rt) for 12 hours (hr) to afford compound 6 in quantitative yield, as described below.

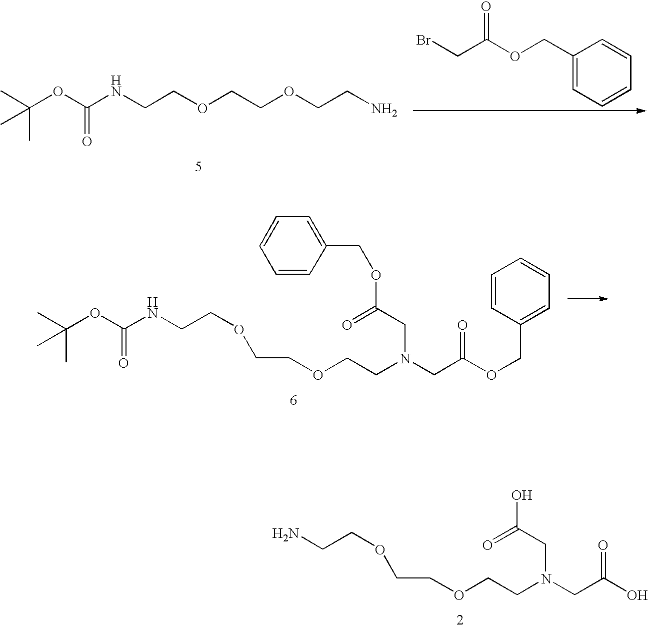

Compound 6, (benzyloxycarbonylmethyl-{2-[2-(2-tert-butoxycarbonylamino-ethoxy)-ethoxy]-ethyl}-amino)-acetic acid benzyl ester (C₂₉H₄₀N₂O₈) was synthesized using the following procedure: to a solution of compound 2, {2-[2-(2-amino-ethoxy)-ethoxy]-ethyl}-carbamic acid tert-butyl ester, (1.00 g, 4.03 mmol) and di(isopropyl)ethylamine (1.50 g, 11.6 mmol) in THF:H₂O (1:1 v/v, 100 mL) was added (rt) a solution of benzyl 2-bromoacetate (2.31 g, 10.1 mmol) in THF:H₂O (1:1 v/v, 100 mL). The resulting solution was stirred (rt) for 12 hours followed by dilution with aqueous acetic acid (5% v/v) and ethylacetate. The organic layer was collected, dried (Na₂SO₄) and concentrated in vacuo to afford a crude oil. A purified sample of this material was prepared by flash column chromatography (SiO₂, eluent gradient of of 8:1 v/v to 3:1 v/v of hexanes:ethyl acetate) to afford (benzyloxycarbonylmethyl-{2-[2-(2-tert-butoxycarbonylamino-ethoxy)-ethoxy]-ethyl}-amino)-acetic acid benzyl ester as a colorless oil (920 mg, 1.69 mmol): ¹H NMR (500 MHz, CDCl3) δ 7.36-7.32 (m, 10H), 5.15 (br s, 4H), 3.71 (br s, 4H), 3.62 (app t, J=5.0 Hz, 2H), 3.53 (br s, 4H), 3.48 (app t, J=5.0 Hz, 2H), 3.28 (app t, J=5.0 Hz, 2H), 3.01 (app t, J=5.0 Hz, 2H), 1.46 (br s, 9H); MS (EI) m/z 545.5 (MH+, 100%).

Compound 2, ({2-[2-(2-amino-ethoxy)-ethoxy]-ethyl}-carboxymethyl-amino)-acetic acid (C₁₀H₂ON₂O₆) was synthesized using the following procedure: a solution of (benzyloxycarbonylmethyl-{2-[2-(2-tert-butoxycarbonylamino-ethoxy)-ethoxy]-ethyl}-amino)-acetic acid benzyl ester (300 mg, 0.551 mmol) in CH₂Cl₂: TFA: H₂O (10:9:1 v/v/v, 30 mL) was stirred (rt) for 30 min. then concentrated in vacuo. The resulting viscous oil was diluted with MeOH (5 mL) and this solution was added in the absence of oxygen to neat 10% Pd/C under a nitrogen atmosphere. The nitrogen atmosphere was displaced with a hydrogen atmosphere (1 atm, ˜1 L balloon) and the suspension was stirred (rt) for 2 h. The resulting suspension was filtered (Celite, MeOH wash) and the filtrate was concentrated in vacuo to afford a colorless oil (280 mg). Residual benzyl alcohol present in this oil was removed by trituration (isopropanol:diethyl ether). This afforded ({2-[2-(2-amino-ethoxy)-ethoxy]-ethyl}-carboxymethyl-amino)-acetic acid as a colorless oil 135 mg, 0.511 mmol): ¹H NMR (500 MHz, CD₃OD) d 3.98 (br s, 4H), 3.84 (app t, J=5.0 Hz, 2H), 3.75 (app t, J=5.0 Hz, 2H), 3.69 (m, 4H), 3.40 (app t, J=5.0 Hz, 2H), 3.16 (app t, J=5.0 Hz, 2H); ¹³C NMR MHz, CD₃OD) d 170.2, 71.3, 71.2, 67.9, 66.6, 57.4, 56.3, 40.6; MS (EI) m/z 265.3 (MH+, 100%), m/z 528.9.

### Example 2

### Synthesis of a C-D-E Compound

The preparation of compound 8, [2-(9H-fluoren-9-ylmethoxycarbonylamino)-ethyl]-carbamic acid 4-nitro-phenyl ester, a moderately stable, crystalline isocyanate equivalent, was performed according to the general method of Liskamp, et al. (Boeijen, A, Ameijde, J. v., Liskamp, R. M. J., J. Org. Chem. 2001, 66, pp 8454-8562) involving the reaction of Fmoc-protected ethylene diamine, 7, with p-nitrophenyl chloroformate (CHCl₃/DIEA).

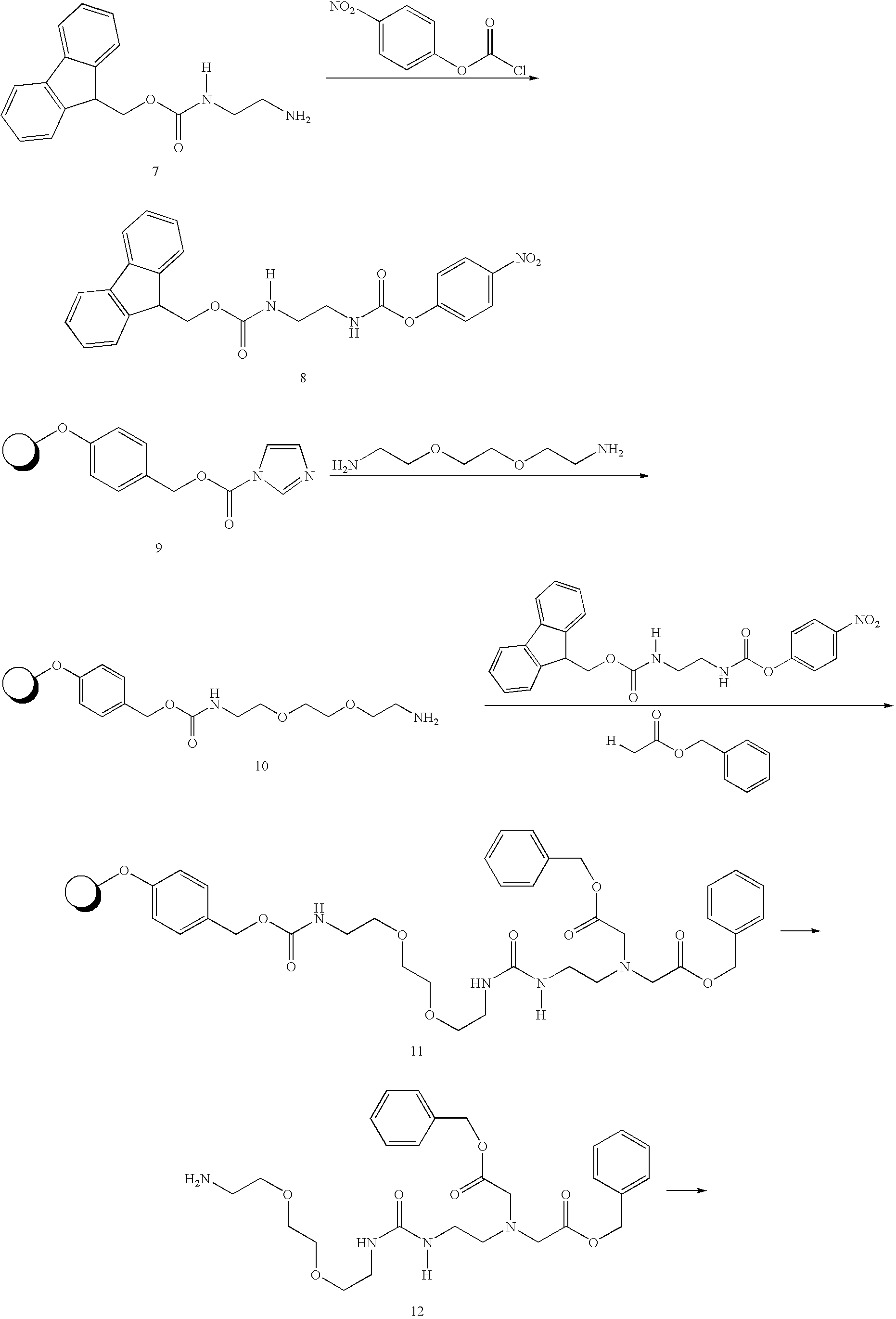

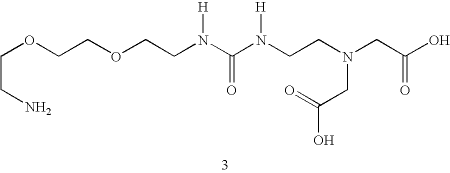

The following method was performed to synthesize compound 12, {[2-(3-{2-[2-(2-amino-ethoxy)-ethoxy]-ethyl}-ureido)-ethyl]-benzyloxycarbonyl methyl-amino}-acetic acid benzyl ester (C₂₇H₃₈N₄O₇): using schlenk-type glassware fitted with gas and vacuum lines, polymer-bound carbonylimidazole Wang-type resin (Aldrich Inc., ˜0.5 mmol/g load level, 5.00 g, ˜2.5 mmol) was treated (rt, 10 min) with CH₂Cl₂ (50 ml) followed by filtration in vacuo. This process was repeated three times.

The resulting swollen and rinsed resin was washed with NMP (50 mL, twice) followed by addition of a solution of 2,2′(ethylenedioxy)bis(ethylamine) in NMP (1.6 M, 25 mL). The resin was gently and orbitally agitated (rt, 12 h) then filtered and the resin washed with NMP (3×50 mL) followed by CH₂Cl₂ (3×50 mL). The washed resin was dried in vacuo for storage. An aliquot of this resin tested positive by Kaiser analysis with ninhydrin while an aliquot of starting resin was negative by Kaiser in side by side tests. A half portion of this primary amine loaded resin (w/w, 2.60 g, theor. loading of ˜1.25 mmol) was treated (rt, 10 min.) with CH₂Cl₂ (50 ml) followed by filtration in vacuo. This process was repeated three times.

The resulting swollen and rinsed resin was washed with NMP (50 mL, twice) followed by addition of a solution of di(isopropyl)ethyl amine in NMP (2.0 M, 4.3 mL) followed by addition of a solution of [2-(9H-fluoren-9-ylmethoxycarbonylamino)-ethyl]-carbamic acid 4-nitro-phenyl ester in NMP (0.20 M, 18 mL) which was prepared according to the general method of Liskamp et al. (Boeijen, A, Ameijde, J. v., Liskamp, R. M. J., J. Org. Chem. 2001, 66, pp 8454-8562). The resulting suspension was gently and orbitally agitated (rt, 2 h) then filtered and the resin washed with NMP (3×50 mL) followed by CH₂Cl₂ (3×50 mL). The washed resin was dried in vacuo for storage. An aliquot of this resin tested negative by Kaiser analysis. This resin was divided in half by weight and one portion was used for the following procedure. This resin portion (˜1.3 g, theor. loading of ˜0.63 mmol) was treated (rt, 10 min.) with CH₂Cl₂ (25 ml) followed by filtration in vacuo and this process was repeated three times.

The resulting swollen and rinsed resin was washed with NMP (25 mL, twice) followed by addition of a solution of piperidine in NMP (20% v/v, 25 mL). The resulting suspension was gently and orbitally agitated (rt, 20 min.) then filtered and the resin was washed with NMP (4×25 mL). To this resin was added a solution of di(isopropyl)ethyl amine in NMP (2.0 M, 5 mL) followed by addition of a solution of benzyl 2-bromoacetate in NMP (1.0 M, 5 mL). The resulting suspension was gently and orbitally agitated (rt). After 10 minutes, a Kaiser test performed on an aliquot of filtered and washed (CH₂Cl₂) resin material tested negative, relative to a side-by-side aliquot of the precursor resin as a positive control, and thus indicating complete dialkylation. After 40 min. total elapsed reaction time, the remainder of the resin material was filtered and the resin washed with NMP (4×50 mL) followed by CH₂Cl₂ (4×50 mL).

The resulting moist resin was treated with CH₂Cl₂ (20 mL) followed by a solution of TFA:H₂O (9:1 v/v, 20 mL) and the resulting suspension was gently and orbitally agitated (rt, 1 h). The resulting bright red resin suspension was filtered, washed with CH₂Cl₂ (2×20 mL) and the combined filtrates were collected and concentrated in vacuo to afford an oil (200 mg). Flash column chromatography (SiO₂, with triethylamine:ethyl acetate:methanol, 6:47:47 v/v/v) afforded {[2-(3-{2-[2-(2-Amino-ethoxy)-ethoxy]-ethyl}-ureido)-ethyl]-benzyloxycarbonylmethyl-amino}-acetic acid benzyl ester as a colorless oil (150 mg, 0.283 mmol, ˜45% overall yield from the starting polymer-bound carbonylimidazole Wang-type resin): MS (EI) m/z 531.5 (MH+, parent ion also a characteristic fragment ion is observed at 441 which may correspond to ionization-induced loss of one benzylic group).

The following method was used to synthesize compound 3, {[2-(3-{2-[2-(2-amino-ethoxy)-ethoxy]-ethyl}-ureido)-ethyl]-carboxymethyl-amino}-acetic acid (C₁₃H₂₆N₄O₇). A solution of {[2-(3-{2-[2-(2-amino-ethoxy)-ethoxy]-ethyl}-ureido)-ethyl]-benzyloxycarbonylmethyl-amino}-acetic acid benzyl ester (75 mg, 0.14 mmol) was diluted with MeOH (10 mL) and this solution was added (in the absence of oxygen) to neat 10% Pd/C under a nitrogen atmosphere. The nitrogen atmosphere was displaced with a hydrogen atmosphere (1 atm, ˜1 L balloon) and the suspension was stirred (rt) for 40 minutes. The resulting suspension was filtered (Celite, MeOH wash) and the filtrate was concentrated in vacuo to afford a colorless oil (68 mg). The only significant contaminant observed was benzyl alcohol. Purification by HPLC (reverse phase column, 0.1% v/v acetic acid in a binary solution of CH₃CN: H₂O, with an elution gradient of ˜5% to ˜95% CH₃CN over ca. 15 minutes) afforded an analytically pure sample of {[2-(3-{2-[2-(2-amino-ethoxy)-ethoxy]-ethyl}-ureido)-ethyl]-carboxymethyl-amino}-acetic acid as a colorless oil (6.5 mg, 0.019 mmol): ¹H NMR (500 MHz, CD₃OD) δ 3.80-3.48 (m, 18H), 3.18 (app t, J=5.0 Hz, 2H); ¹³C NMR (125 MHz, CD₃OD) δ 170.5, 161.5, 71.3, 71.2, 67.8, 66.6, 58.7, 58.0, 41.2, 40.7, 36.6; MS (EI) m/z 351.4 (MH+, 100%).

### Example 3

### Synthesis of an A-B′—C′-D-E Compound with a Fluorescent Group

Using procedures described in the literature, C-D-E compound 13 was coupled to compound 14 to yield 15 (Tegge, W. et al., Analytical Biochem. 1999, 276, pp. 227-241).

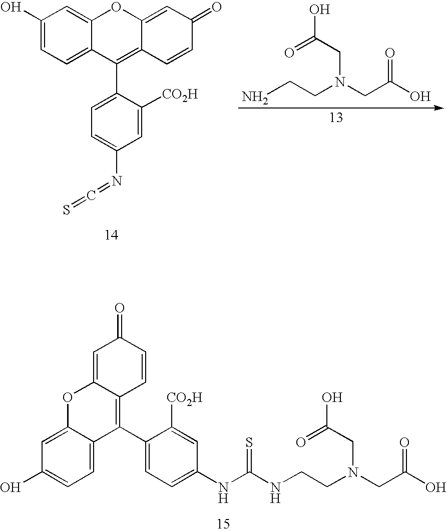

### Example 4

### Synthesis of an A-B′—C′-D-E Compound with a Fluorescent Group

Fourteen micromoles of compound 16 in 0.25 mL H₂O was adjusted to pH 10.5 with 0.2 M NaOH. To this, 15 micromoles of compound 17 in 0.135 mL was added in 25 micoliter aliquots with a 10 min incubation between additions. After each incubation period, the pH was readjusted to approximately 10.5. After the last addition, the reaction was allowed to incubate overnight at room temperature. 1 M HCl was added dropwise until the product precipitated at pH 2.5. The precipitate was collected by centrifugation at 12,000×g/5 min and washed with ethanol. The supernatant was collected and again precipitated and the pellet was washed with ethanol. Both pellets in ethanol were pooled and lyophilized overnight. The pellets were resuspended in the aqueous solution and titrated to pH 7.0. A 3-fold excess of FeCl₃ was added and the precipitate was collected by centrifugation. The pellet was washed with water and the precipitate was pelleted by centrifugation. The washing and centrifugation steps were repeated five times. The washed pellet was resuspended in DMSO. The coupling of compound 16 to compound 17 was followed by mass spectrometry. Compound 16 has a m/z=265.2 in the M+H state with some 2M+H observed with a m/z=528.9. Compound 17 has a m/z=509.6 in the M+H state. Compound 18 has a m/z=657.5 in the M+H state.

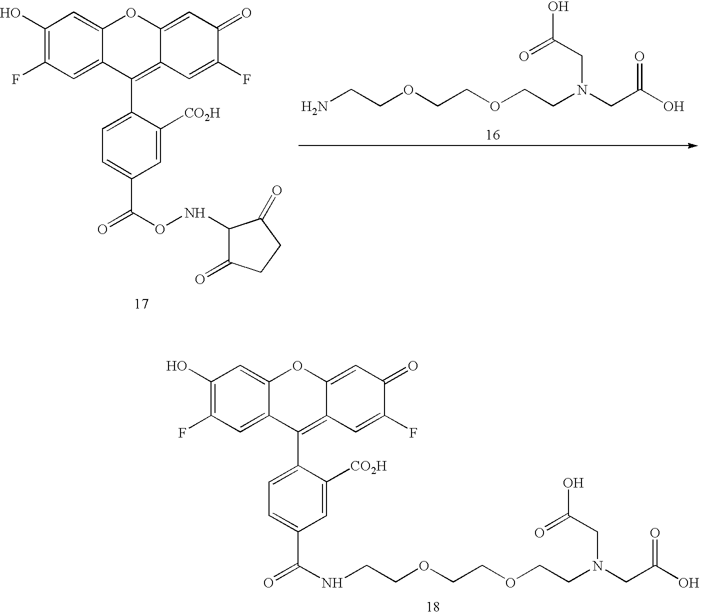

### Example 5

### Synthesis of an A-B Compound

The A-B components were synthesized using methods known in the literature, for example, the methods described in J. Org. Chem. 1988, 53(15), 3521-3529, Tet. Lett. 1998, 39, pp. 1573-1576, and Zeitsobrift fucr. Natuirforschung, B: chemical sciences, 1988, 43(3), 361-367. Compound 19 was treated with Ln and then reacted with 2-(chloromethyl)pyridine-4-carboxylic acid via an Ullman-type coupling to arrive at compound 20.

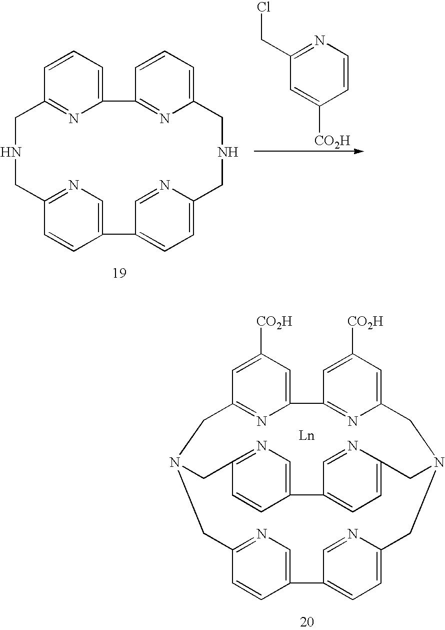

### Example 7

### Qualitative Identification of Phosphophorylated Ser/Thr/Tyr Proteins

Proteins to be evaluated for phosphorylation are separated by SDS-PAGE and electro blotted to a PVDF or nitrocellulose membrane. The membrane is incubated for 4 hours at room temperature with Tris (pH 7.8) saline containing 0.2% Tween-20/0.5% polyvinyl alcohol (PVA) (Anal. Biochem. 1999, 276, 129-143; J. Immunol. Methods 1982, 55(3), 297-307) to block non-specific binding sites. The membrane is briefly rinsed with the detergent saline followed by 1% acetic acid/0.1% Tween-20/0.5% PVA. The membrane is then incubated for 3 hours at room temperature with the same solution containing a probe with chelated iron conjugated to a N-hydroxysuccinimidyl ester of AlexaFlour-555 (Molecular Probes, Eugene Oreg.), conjugated as described in Example 4. The membrane is rinsed three times for 15 min with excess 1% acetic acid/0.2% Tween-20/0.5% PVA/5 mM NaH₂PO₄ (pH 5.5) followed by image analysis in 1% acetic acid (pH 5.5) using a Typhoon 9400 imager using DeCyder software (G.E. Health Systems, Pisctaway, N.J.). Once imaged, the probe is stripped from the membranes by washing extensively with 0.2 M Na₃PO₄ (pH 8.4) and reprobed by a standard Western Blotting protocol using an anti-phosphotyrosine antibody (4G10, Upstate Cell Signaling Solutions, Lake Placid N.Y.) conjugated to N-hydroxysuccinimidyl ester of AlexaFlour-647. Subtractive analysis of the imaged gels enables a qualitative identification of phosphotyrosine and phospho-Serine/Threonine containing proteins in the same gel regions. This approach is used to detect phosphoproteins from native polyacrylamide gels, SDS-polyacrylamide gels, and 2-D.

### Example 8

### Quantitative Identification of Phosphophorylated Ser/Thr/Tyr Proteins

The procedure described in Example 7 is followed. The assay is quantitative with the chelated probe alone since the molar ratio is 1:1 with phosphate and fluorescent probe. The difference mapping of phospho-Ser/Thr and phospho-Tyr is quantitative if the exact molar ratio is determined for the AlexaFlour-647 labeling of the anti-phosphotyrosine monoclonal antibody.

After staining with the probe, protein bands of interest are cut from the PVDF/nitrocellulose membrane, the probe stripped off the membrane, a protease is added for digestion, and the peptides eluted from the membrane (Pappin, D. J. C. et al., In Mass Spectrometry in the Biological Sciences; Burlingame, A. L., Carr, S. A., Eds.; Humana Press: Totowan N.J., pp. 135-150, 1995). The peptide sample is then evaluated by mass spectrometry (Id.; Anal. Chem. 1996, 68, 850-858; Anal. Biochem. 1999, 276, 129-143).

### Example 9

### Quantitative Identification of Phosphophorylated Ser/Thr/Tyr Proteins with Gel Detection

The procedures described in Example 8 are followed. Proteins to be evaluated for phosphorylation are separated on SDS-PAGE or Native-PAG and fixed in 50% methanol/5% acetic acid. The gel is allowed to equilibrate with a solution (1% acetic acid, pH 5.5) containing an chelated probe conjugated to a fluorescent dye. Excess probe is washed out of the gel by agitating the gel with many changes of an excess volume of 1% acetic acid/5 mM NaH₂PO₄ (pH 5.5). The bands of interest are identified and imaged. Protein bands of interest are excised from the gel, the probe is eluted from the embedded protein, and the protein is digested and identified by mass spectrometry as described above.

### Example 10

### Kinase Inhibitor Assay

In a 96-well plate, 100 μl of the following assay medium is added: 50 mM Hepes (pH 7.5), 100 mM NaCl, 2 mM DTT, 1% BSA, 100 mM MgCl₂, 100 mM MnCl₂, a nonomeric peptide tagged with biotin at the N-terminus, ATP, test inhibitors, and a kinase. The kinase substrate and ATP are then added at concentrations incremental to the Kₘ values. The distance between the biotin affinity tag and the phosphorylation site is 6 residues. The assay reaction contains molecules to be tested for kinase inhibitor properties by titrating from a stock solution of DMSO to a 3 μM final concentration. The assay media with and without inhibitors are incubated for 90 minutes.

The enzyme reactions are quenched by addition of a quench buffer containing 0.2 M EDTA and 0.1 M KF. APC-streptavidin is added and the solutions are incubated for 90 minutes. Acid is added to reduce the pH to 4 followed by the addition of compound a europium cryptate conjugated probe according to the invention. The Eu—Fe³⁺-probe:ATP ratio is predetermined since some nonspecific binding to ATP occurs. The detection reagents are incubated for 6 hours. Specific FRET is read at both 665 nm and 620 nm using a RubyStar reader. Specific FRET is expressed as % AF as follows:

$$\frac{665 nm \text{/} 620 nm ( Sample ) - 665 nm \text{/} 620 nm ( Blank )}{665 nm \text{/} 620 nm ( Control ) - 665 nm \text{/} 620 nm ( Blank )} \times 100 = \% Δ F = \% Inhibition$$

### Example 11

### Identification and Quantification of Poyhistidine Tagged Proteins

The method is performed as described in Example 8 except the fluorescent metal chelating probe is coordinated with Ni²⁺ or Co²⁺ and the binding step is performed in 50 mM HEPES (pH 8.0), 2 mM imidazole, 0.15 M NaCl, 1 mM BME (binding buffer). The proteins are imaged as described above. The probe is eluted from the bands using 60 mM imidazole in the binding buffer. Protein identification is performed as described above.

### Example 12

### Phosphatase HTRF Assay

Biotinylated phosphorylated peptide (EGFR 988-998) is mixed with PTP1B in a final volume of 150 ul of 50 mM HEPES (pH 7.5), 1 mM DTT, 25 mM NaCl, 0.1% NP-40 to give an optimal enzyme concentration and substrate at or near the previously determined Km. The reactions are quenched with a final of 1% acid at the desired time and the samples are processed for FRET by HTRF as described above.

What is claimed is:

1. A compound of the formula:
C-D-E
wherein (C) is a coupling group, (E) is a chelating group, and (D) is a linker group chosen from:
—(CH₂)ₘ(OCH₂CH₂)ₙ—O—(CH₂)ₚ—(O)_{q}-Z-(CH₂)ᵣ—;
—(CR¹R²)ₘ-[(CR³R⁴)ₚ—(O)_{q}]ₙ-Z-(CR⁵R⁶)ᵣ—;
—(CH₂)ₘ-[(CR¹R²)ₚ—(O)_{q}]ₙ-Z-(CH₂)ᵣ—;
—(CH₂)ₘ—(C₆R¹R²R³R⁴)ₙ—(CH₂)ᵣ—; and
—(CH₂)ₘ—(CR¹R²CR³R⁴NR⁵)ₙ—(CH₂)ₚ-Z-(CH₂)ᵣ—;
wherein Z is a urea group or is absent;
m ranges from 0 to 3;
n ranges from 0 to 170;
p ranges from 0 to 3;
q is 0 or 1;
r ranges from 0 to 3; and
R¹, R², R³, R⁴, R⁵ and R⁶ are each independently chosen from hydrogen, fluorine, and C₁-C₆ alkyl,
provided that when Z is absent, n is 0, and the chelating group (E) is of the formula: 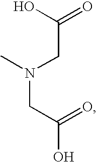
then m, p and q are each not 2.

2. The compound of claim 1, wherein the coupling group (C) is chosen from an amino group, an aldehyde group, an alkyl halide group, a thiol group, and a hydroxy group.

3. The compound of claim 1, wherein the coupling group (C) is a secondary amino group.

4. The compound of claim 3, wherein the coupling group (C) has the structure —NHR′ wherein R′ is a C₁-C₆ alkyl group.

5. The compound of claim 1, wherein the coupling group (C) is —NH₂.

6. The compound of claim 1, wherein n ranges from 1 to 20.

7. The compound of claim 6, wherein n ranges from 1 to 5.

8. The compound of claim 1, wherein at least one of m, p, and q ranges from 1 to 3.

9. The compound of claim 1, wherein the sum of m, n, p, and r ranges from 0 to 170.

10. The compound of claim 1, wherein Z is a urea group of the formula —NHC(O)NH— or —CH₂CH₂NHC(O)NH—.

11. The compound of claim 1, wherein the compound is of the formula:

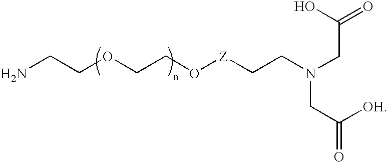

12. The compound of claim 11, wherein Z is a urea group of the formula —CH₂CH₂NHC(O)NH—.

13. The compound of claim 11, wherein Z is absent.

14. The compound of claim 1, wherein the chelating group (E) is chosen from an imidazo group, a hydroxamic acid group, a hydroxylamine group, and a sulfonic acid group.

15. The compound of claim 1, wherein the chelating group (E) is of the formula: 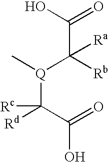
wherein Q is chosen from N, P, and CH, and R^{a}, R^{b}, R^{c}, and R^{d} are each independently chosen from hydrogen, fluorine, and C₁-C₆ alkyl.

16. The compound of claim 15, wherein R^{a}, R^{b}, R^{c}, and R^{d} are each hydrogen.

17. The compound of claim 15, wherein Q is N.

18. The compound of claim 17, wherein R^{a}, R^{b}, R^{c}, and R^{d} are each hydrogen.

19. The compound of claim 1, wherein the chelating group (E) is of the formula: 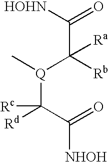
wherein Q is chosen from N, P, and CH; and R^{a}, R^{b}, R^{c}, and R^{d} are each independently chosen from hydrogen, fluorine, and C₁-C₆ alkyl.

20. The compound of claim 19, wherein R^{a}, R^{b}, R^{c}, and R^{d} are each hydrogen.

21. The compound of claim 1, wherein the chelating group (E) is of the formula: 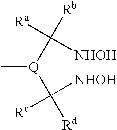
wherein Q is chosen from N, P, and CH; and R^{a}, R^{b}, R^{c}, and R^{d} are each independently chosen from hydrogen, fluorine, and C₁-C₆ alkyl; or one or both of (R^{a} and R^{b}) and (R^{c} and R^{d}) together form a carbonyl group.

22. The compound of claim 21, wherein R^{a}, R^{b}, R^{c}, and R^{d} are each hydrogen.

23. The compound of claim 1, wherein the chelating group (E) is of the formula: 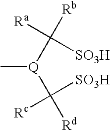
wherein Q is chosen from N, P, and CH, and R^{a}, R^{b}, R^{c}, and R^{d} are each independently chosen from hydrogen, fluorine, and C₁-C₆ alkyl; or one or both of (R^{a} and R^{b}) and (R^{c} and R^{d}) together form a carbonyl group.

24. The compound of claim 23, wherein R^{a}, R^{b}, R^{c}, and R^{d} are each hydrogen.

25. The compound of claim 1, wherein the chelating group (E) is of the formula: 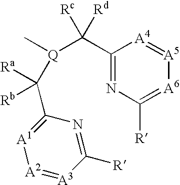
wherein Q is chosen from N, P, and CH;
R^{a}, R^{b}, R^{c}, and R^{d} are each independently chosen from hydrogen, fluorine, and C₁-C₆alkyl; and
A¹, A², A³, A⁴, A⁵, and A⁶ are each independently chosen from N and C—R′, wherein each R′ is chosen from hydrogen, fluorine and C₁-C₆ alkyl.

26. The compound of claim 25, wherein Q is N; R^{a}, R^{b}, R^{c}, and R^{d} are each hydrogen; and A¹, A², A³, A⁴, A⁵ and A⁵ are each CH.

27. The compound of claim 1, wherein the chelating group (E) is of the formula: 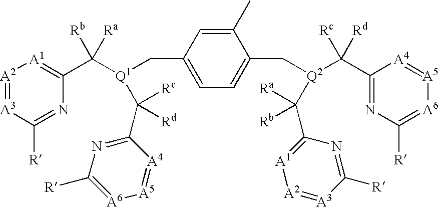
wherein Q¹ and Q² are each independently chosen from N, P, and CH;
R^{a}, R^{b}, R^{c}, and R^{d} are each independently chosen from hydrogen, fluorine, and C₁-C₆alkyl; and
A¹, A², A³, A⁴, A⁵, and A⁶ are each independently chosen from N and C—R′, where R′ is chosen from hydrogen, fluorine and C₁-C₆ alkyl.

28. The compound of claim 27, wherein Q¹ and Q² are each N, R^{a}, R^{b}, R^{c}, and R^{d} are each hydrogen, and A¹, A², A³, A⁴, A⁵, and A⁶ are each CH.

29. The compound of claim 1, wherein (D) is
—(CH₂)ₘ(OCH₂CH₂)ₙ—O—(CH₂)ₚ—(O)_{q}-Z-(CH₂)ᵣ—.

30. The compound of claim 1, wherein (D) is
—(CR¹R²)ₘ-[(CR³R⁴)ₚ—(O)_{q}]ₙ-Z-(CR⁵R⁶)ᵣ—.

31. The compound of claim 30, wherein R¹, R², R³, R⁴, R⁵ and R⁶ are each hydrogen.

32. The compound of claim 1, wherein (D) is
—(CH₂)ₘ-[(CR¹R²)ₚ—(O)_{q}]ₙ-Z-(CH₂)ᵣ—.

33. The compound of claim 32, wherein R¹ and R² are each hydrogen.

34. The compound of claim 1, wherein (D) is
—(CH₂)ₘ—(C₆R¹, R²R³R⁴)ₙ—(CH₂)ᵣ—.

35. The compound of claim 34, wherein R¹, R², R³, and R⁴ are each hydrogen.

36. The compound of claim 1, wherein (D) is
—(CH₂)ₘ—(CR¹, R²CR³R⁴NR⁵)ₙ—(CH₂)ₚ-Z-(CH₂)ᵣ—.

37. The compound of claim 36, wherein R⁵ is hydrogen.

38. The compound of claim 36, wherein R¹, R², R³, and R⁴ are each hydrogen.

39. The compound of claim 38, wherein R⁵ is hydrogen.

40. A compound of the formula:

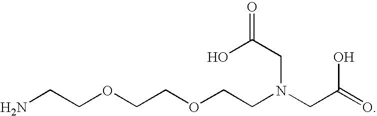

41. A compound of the formula:

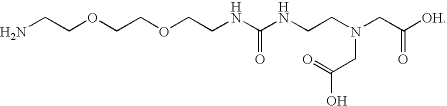

42. A compound of the formula: [chemical structure]
